(19) United States
(12) Patent Application Publication
(10) Pub. No.: US 2025/0134693 A1
(43) Pub. Date: May 1, 2025

(54) OSTOMY BAG AND RELATED CONNECTION MECHANISM

(71) Applicant: BOSTON SCIENTIFIC SCIMED, INC., Maple Grove, MN (US)

(72) Inventors: Cristina ROMANY, Orlando, FL (US); Allison KUMARASENA, Wellesley, MA (US); Rahul PRABHU, Brighton, MA (US); Laura Emily SABBAN, Norton, MA (US); Kurt Nicholas ROBAKIEWICZ, Upton, MA (US); Lisa Margaret SANGREE, Boston, MA (US); Julia VEITCH, Waltham, MA (US); Colin BUCKLEY, Brookline, MA (US)

(21) Appl. No.: 18/918,953

(22) Filed: Oct. 17, 2024

Related U.S. Application Data
(60) Provisional application No. 63/593,870, filed on Oct. 27, 2023.

(51) Int. Cl. A61F 5/443; A61F 5/445; A61F 5/448

(52) U.S. Cl. CPC A61F 5/443; A61F 5/448; A61F 2005/4455

(57) ABSTRACT

An ostomy device includes an implant shaped in a ring and sized for placement in or adjacent to a hole in an abdominal wall, the implant including a surface for contacting a lumen. The implant includes a first part including the surface for contacting the lumen, the first part, when placed in or adjacent to the hole in the abdominal wall, extending in a direction orthogonal to the abdominal wall to support the lumen. The implant includes a region for attaching an ostomy bag.

### PRIORITY CLAIM

The present disclosure claims priority to U.S. Provisional Patent Application Ser. No. 63/593,870 filed Oct. 27, 2023; the disclosure of which is incorporated herewith by reference.

### FIELD

The present disclosure relates to ostomy devices for post-procedural ostomy management.

### BACKGROUND

An ostomy generally refers to a surgical procedure in which an opening, or stoma, is created in the abdomen to provide a path for human waste or a stool (e.g., a liquid waste, a solid waste, and/or a gas waste) to be removed from the body. Sections of the gastrointestinal tract are bypassed, and the waste typically produced and transported through the body is excreted through the stoma. Traditionally, ostomies are managed through technology such as an ostomy bag, which the patient must regularly vacate and replace. Ostomy patients may experience complications with the stoma including skin irritation, stoma leaks, bleeding and other issues. Additionally, the stoma bypass may alter in size from patient to patient and may vary for a given patient over time.

### SUMMARY

The present disclosure relates to an ostomy device which includes an implant shaped in a ring and sized for placement in or adjacent to a hole in an abdominal wall, the implant including a surface for contacting a lumen. The implant includes a first part including the surface for contacting the lumen, the first part, when placed in or adjacent to the hole in the abdominal wall, extending in a direction orthogonal to the abdominal wall to support the lumen. The implant includes a region for attaching an ostomy bag.

In an embodiment, a cross section of the implant comprises a corner at a proximal end of the first part and a second part extending radially outward from the corner.

In an embodiment, to form a stoma, the second part is inserted through the hole in the abdominal wall so that a top surface of the second part is brought into contact with an interior surface of the abdominal wall, the first part extends through the hole and protrudes a distance past the abdominal wall, and the lumen is brought through a center channel of the implant, wherein an inner surface of the first part contacts an outer surface of the lumen.

In an embodiment, the lumen is attached to the first part and the second part is attached to the abdominal wall.

In an embodiment, to form a stoma, the second part is placed on an outer surface of the abdominal wall surrounding the hole in the abdominal wall so that a bottom surface of the second part is brought into contact with the outer surface of the abdominal wall, the first part extends a distance distally from the abdominal wall, and the lumen is brought through a center channel of the implant, wherein an inner surface of the first part contacts an outer surface of the lumen.

In an embodiment, the lumen is attached to the first part and the second part is attached to the abdominal wall.

In an embodiment, a cross section of the implant comprises a corner at a distal end of the first part, a second part extending radially outward from the corner and a distal part extending from the corner.

In an embodiment, to form a stoma, the implant is inserted through the hole in the abdominal wall so that a top surface of the second part is brought into contact with an interior surface of the abdominal wall, the first part extends proximally, the distal part contacts tissue surrounding the hole in the abdominal wall, and the lumen is brought through a center channel of the implant, wherein an inner surface of the first part contacts an outer surface of the lumen and the lumen remains internal to the abdominal wall.

In an embodiment, the lumen is attached to the first part and the second part is attached to the abdominal wall and wherein the distal part comprises a bag attachment region within or adjacent to the hole in the abdominal wall.

In an embodiment, the implant comprises a distal part on a distal end of the first part.

In an embodiment, to form a stoma, the implant is inserted through the hole in the abdominal wall so that the distal part contacts tissue surrounding the hole in the abdominal wall and an inner surface of the lumen is brought around the first part to an inner surface of the abdominal wall.

In an embodiment, the lumen is attached to the first part or to the inner surface of the abdominal wall, wherein the distal part comprises the bag attachment region within or adjacent to the hole in the abdominal wall.

In addition, the present disclosure relates to an adhesive material which includes a first adhesive surface for placement on an area surrounding an entrance opening to an ostomy bag; and a second adhesive surface for placement on a stoma or an implant integrated with a stoma. The adhesive material has multiple concentric circles of perforations to facilitate tearing by a user to size the adhesive material for an anatomy of the user comprising the stoma.

In an embodiment, the perforations provide breathing holes.

In an embodiment, the adhesive material further includes multiple breathing holes disposed between the circles of perforations.

In addition, the present disclosure relates to a method for implanting an ostomy device. The method includes placing an implant of the ostomy device in or adjacent to a hole in an abdominal wall, the implant shaped in a ring and including a surface for contacting a lumen, the implant including a first part including the surface for contacting the lumen, the first part, when placed in or adjacent to the hole in the abdominal wall, extending in a direction orthogonal to the abdominal wall to support the lumen, the implant comprising a region for attaching an ostomy bag; and attaching the lumen to the implant to form a stoma.

In an embodiment, a cross section of the implant comprises a corner at a proximal end of the first part and a second part extending radially outward from the corner. To form the stoma, the second part is inserted through the hole in the abdominal wall so that a top surface of the second part is brought into contact with an interior surface of the abdominal wall, the first part extends through the hole and protrudes a distance past the abdominal wall, and the lumen is brought through a center channel of the implant, wherein an inner surface of the first part contacts an outer surface of the lumen. The lumen is attached to the first part and the second part is attached to the abdominal wall.

In an embodiment, a cross section of the implant comprises a corner at a proximal end of the first part and a second part extending radially outward from the corner,

wherein, to form the stoma, the second part is placed on an outer surface of the abdominal wall surrounding the hole in the abdominal wall so that a bottom surface of the second part is brought into contact with the outer surface of the abdominal wall, the first part extends a distance distally from the abdominal wall, and the lumen is brought through a center channel of the implant, wherein an inner surface of the first part contacts an outer surface of the lumen. The lumen is attached to the first part and the second part is attached to the abdominal wall.

In an embodiment, a cross section of the implant comprises a corner at a distal end of the first part, a second part extending radially outward from the corner and a distal part extending from the corner. To form the stoma, the implant is inserted through the hole in the abdominal wall so that a top surface of the second part is brought into contact with an interior surface of the abdominal wall, the first part extends proximally, the distal part contacts tissue surrounding the hole in the abdominal wall, and the lumen is brought through a center channel of the implant, wherein an inner surface of the first part contacts an outer surface of the lumen and the lumen remains internal to the abdominal wall. The lumen is attached to the first part and the second part is attached to the abdominal wall, wherein the distal part comprises the bag attachment region within or adjacent to the hole in the abdominal wall.

In an embodiment, the implant comprises a distal part on a distal end of the first part. To form the stoma, the implant is inserted through the hole in the abdominal wall so that the distal part contacts tissue surrounding the hole in the abdominal wall and an inner surface of the lumen is brought around the first part to an inner surface of the abdominal wall. The lumen is attached to the first part or to the inner surface of the abdominal wall, wherein the distal part comprises the bag attachment region within or adjacent to the hole in the abdominal wall.

### BRIEF DESCRIPTION

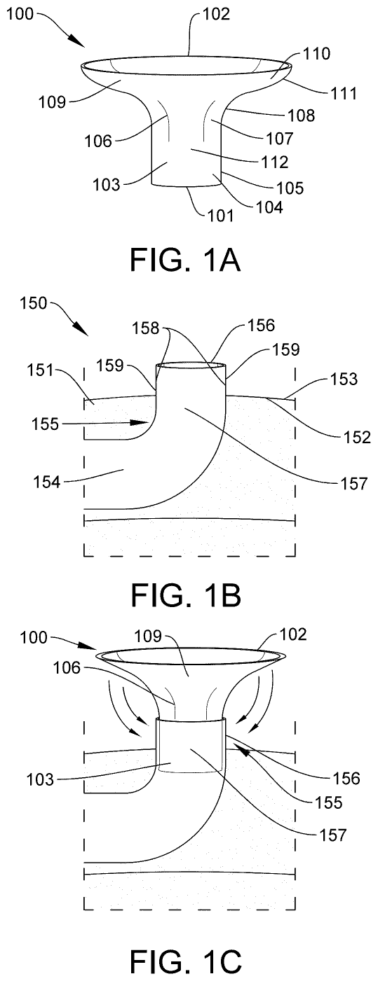

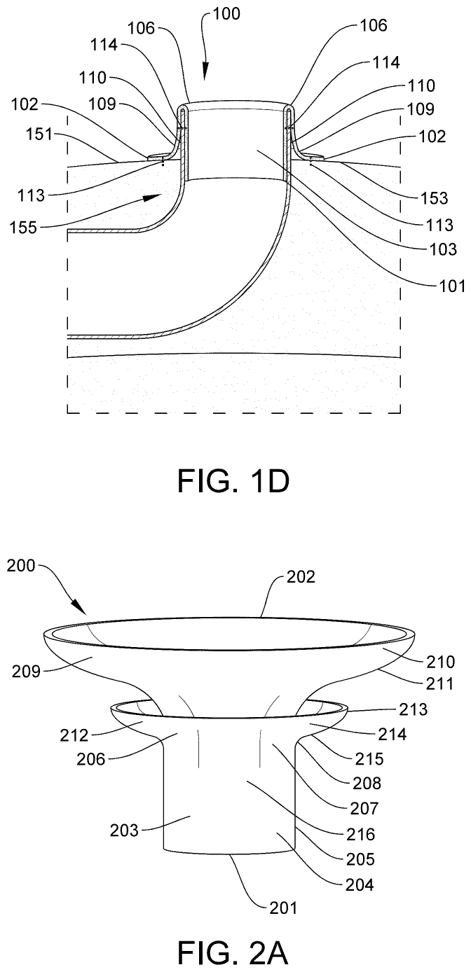

FIG. 1a shows an implant comprising a body and a flared portion that can be inverted during deployment of the implant according to one embodiment.

FIG. 1b shows a patient anatomy including a stoma extending from the intestines and protruding through the abdominal wall.

FIG. 1c shows the implant of FIG. 1a in an insertion state inserted into the stoma.

FIG. 1d shows the implant of FIG. 1a in a deployment state with the flared portion of the implant inverted.

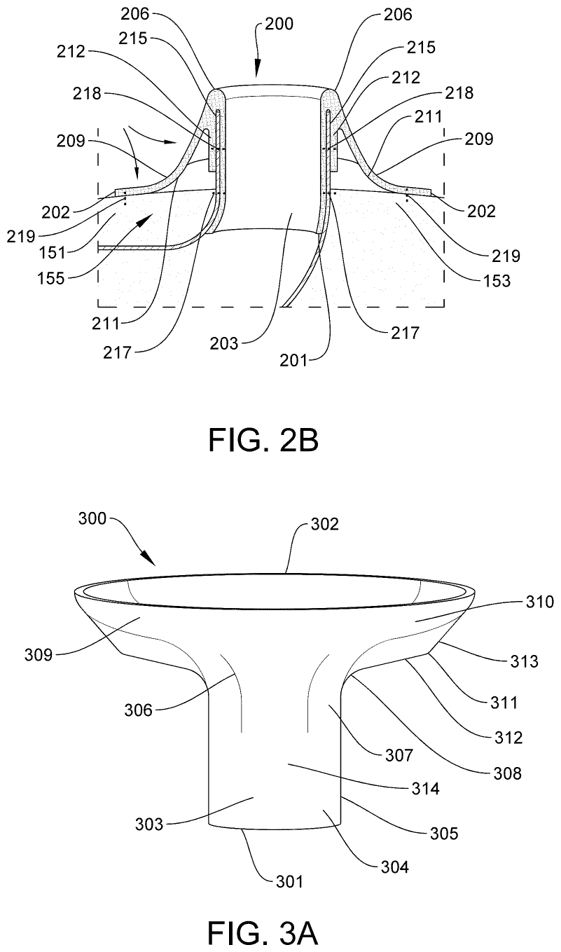

FIG. 2a shows an implant comprising a body, a first flared portion and a second flared portion that can be inverted during deployment of the implant according to another embodiment.

FIG. 2b shows the implant of FIG. 2a in a deployment state with the first flared portion and the second flared portion of the implant inverted.

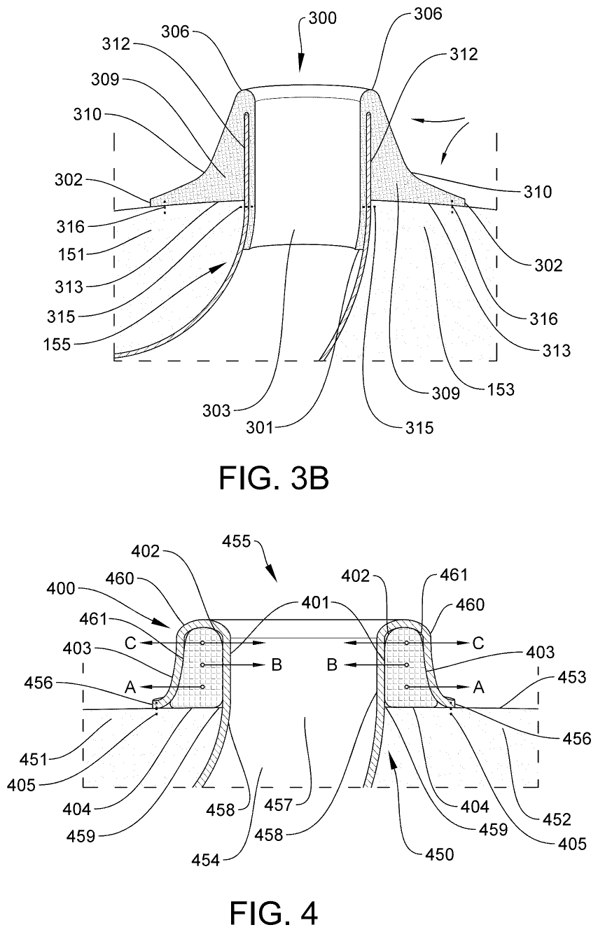

FIG. 3a shows an implant comprising a body and a flared portion shaped to fit snugly into the area adjacent to the stoma and the skin when the flared portion is inverted according to another embodiment.

FIG. 3b shows the implant of FIG. 3a in a deployment state with the flared portion of the implant inverted.

FIG. 4 shows an implant for providing a surface to form a stoma according to one embodiment.

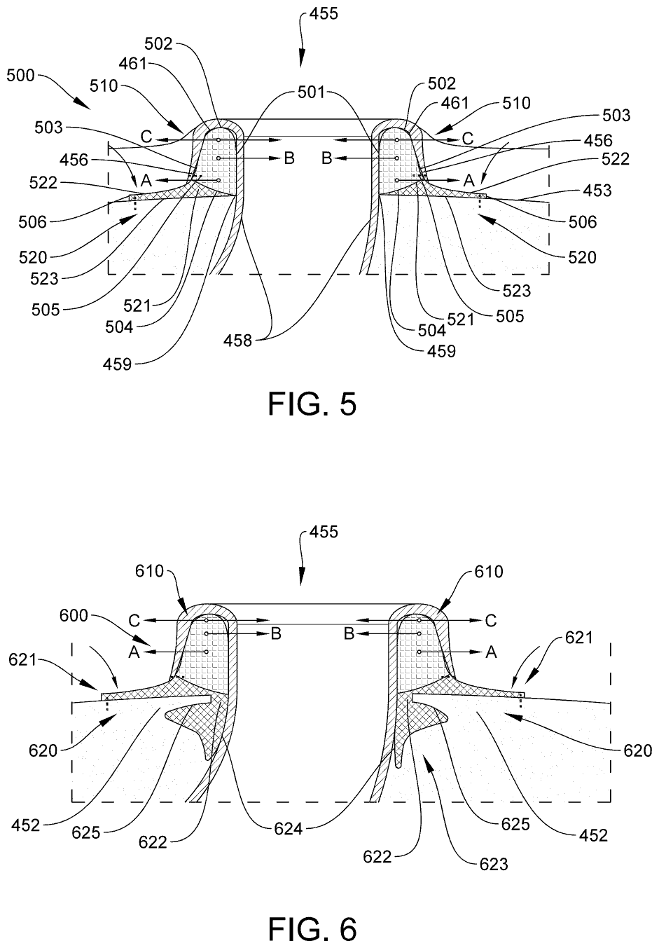

FIG. 5 shows an implant including a body for providing a surface to form a stoma and a skin barrier external to the abdominal wall according to another embodiment.

FIG. 6 shows an implant including a body for providing a surface to form a stoma and a skin barrier both internal and external to the abdominal wall according to still another embodiment.

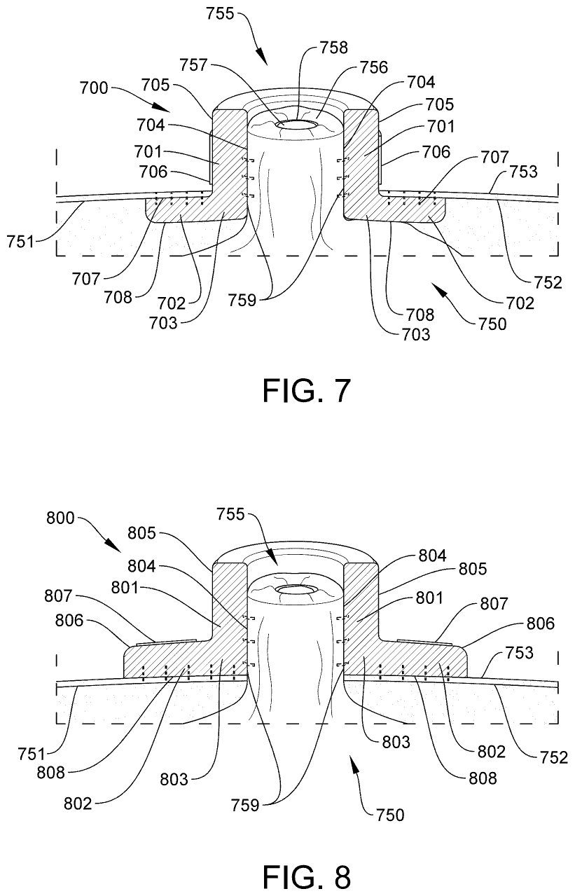

FIG. 7 shows an implant providing a scaffold partially external and partially internal to the abdominal wall for supporting a stoma according to one embodiment.

FIG. 8 shows an implant providing a scaffold fully external to the abdominal wall for supporting a stoma according to another embodiment.

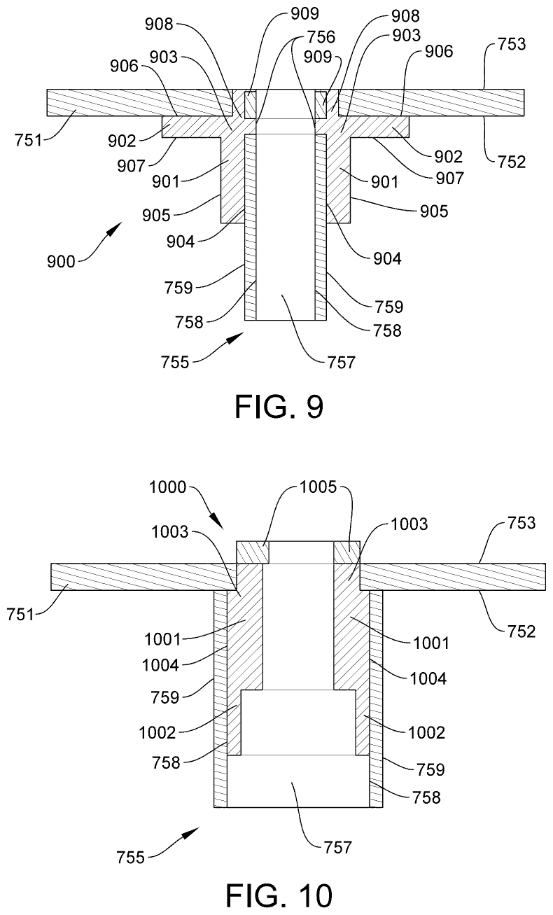

FIG. 9 shows an implant providing a scaffold internal to the abdominal wall for supporting a stoma according to yet another embodiment.

FIG. 10 shows an implant providing a scaffold internal to the abdominal wall and internal to the lumen for supporting a stoma according to yet another embodiment.

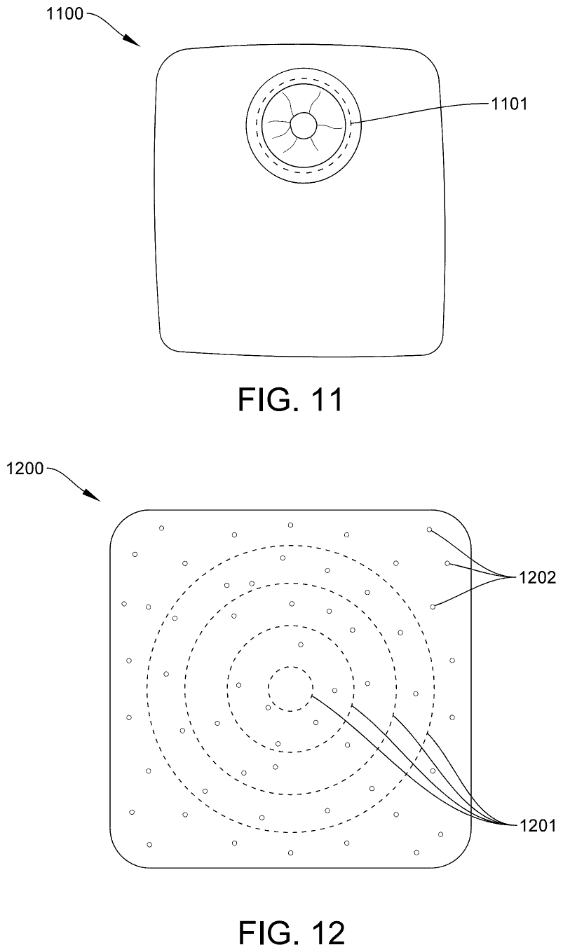

FIG. 11 shows an ostomy bag including a region for interfacing with an implant according to one or more of the previous embodiments.

FIG. 12 shows an adhesive material including a number of rings composed of perforations in the material.

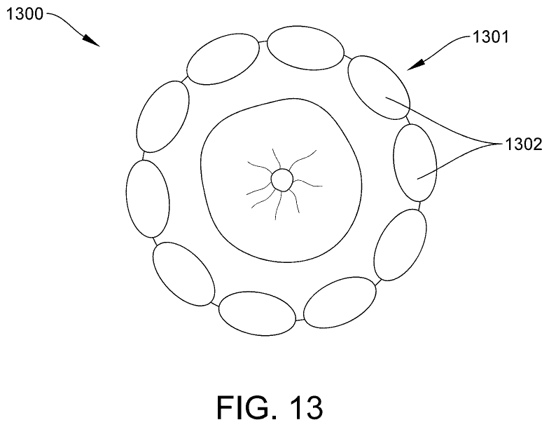

FIG. 13 shows a diagram of ferrous metal beads in a ring around a stoma.

### DETAILED DESCRIPTION

The present disclosure may be further understood with reference to the following description and the appended drawings, wherein like elements are referred to with the same reference numerals. The present disclosure relates to ostomy devices for post-procedural ostomy management.

It is noted that the terms proximal and distal as used herein refer to the anatomy of the patient wherein “proximal” refers to a direction toward the interior of the body and “distal” refers to a direction toward the exterior of the body. With regard to the exemplary ostomy devices described herein, a proximal side of the device generally refers to the side that interfaces with the intestine and a distal side of the device generally refers to the side that interfaces with the environment exterior to the patient.

There are several types of ostomy procedures including a colostomy, an ileostomy and a urostomy. In a colostomy, portions of the large intestine (e.g., colon and/or rectum) are removed and the remaining tissue is brought through an opening in the abdomen to form a stoma. In an ileostomy, the ileum (end of the small intestine) is brought through the abdomen to form the stoma. After any of these procedures is performed, waste can be passed through the stoma, e.g., into an ostomy bag. In a urostomy, a portion of an internal organ (e.g., a portion of the small intestine) is detached from the rest of the organ (e.g., after removal of the urinary bladder) and brought through the abdomen to form a stoma. This detached portion of the small intestine is then coupled to the ureters to allow for the passage of urine through the detached portion of the small intestine and out of the stoma to an ostomy bag.

Some ostomy procedures can be performed laparoscopically using small incisions while others are performed as open surgeries using one or more larger incisions. Some ostomy procedures are permanent, e.g., in the case of bowel or bladder cancer or serious injury, while others are reversible, e.g., after the bowel has recovered from an event such as an infection, inflammation or a stab wound.

Typically, post-procedural ostomy management includes the use of an ostomy bag. Such bags are generally attached to the stoma to collect waste which may then be evacuated at appropriate times. Different procedures may require different durations for which the ostomy bag must be used and, as mentioned above, some ostomies are permanent. The ostomy bag can be attached via adhesives to the skin which the patient must cut, adhere, and replace regularly on their own. The challenge with this technology is the difficulties faced by the patient post-procedurally which include infection, leaking, smells, and social pressures.

When patients undergo ileostomies or like procedures, the stoma bypass created may alter in size from patient to patient. The stoma size for a given patient may also vary with time. As a result, current ostomy bags may require resizing and cutting before use or connection to the patient. If the stoma were a predictable, consistent, or of controllable size, this might remove a step in the user's procedures and reduce challenges associated with ostomy bag attachment.

According to various exemplary embodiments, an ostomy device comprises an implant that can be deployed during the ostomy procedure to control the stoma opening size and/or outer diameter. In various embodiments, the ostomy device can be flexible, rigid, expandable, contractable, or a combination thereof. The ostomy device can be formed of a biocompatible material such as, e.g., silicone. In some embodiments, the device contains a slow release medicative compound to prevent infection and/or add moisturization/comfort to the interfacing skin and tissue. In some embodiments, the device may have a porous or microporous surface structure to enable tissue integration for patients who will require the stoma bypass for the rest of their lives. In some embodiments, the device enables the ostomy bag to be connected to the device itself as opposed to a connection of the bag to the surrounding skin to avoid repetitive adhesion of the bag to the skin. In some embodiments, the ostomy device comprises multiple implants used in combination with one another.

In further exemplary embodiments, devices and techniques are described for attaching an ostomy bag to the skin, to the stoma, and/or directly to an implant after the ostomy procedure. In some embodiments, an ostomy bag is designed to attach to one or more of the implants/devices described herein. In some embodiments, an adhesive material for attaching an ostomy bag to a stoma/skin is described including breathing holes and perforations for adjusting the size of the adhesive material. In some embodiments, a magnet is used to form a connection between an ostomy bag and the patient. Depending on medical considerations specific to a given patient, selected ones of the embodiments described herein may be more or less appropriate under given circumstances.

In some embodiments, an implant comprises a tubular body configured for insertion into the stoma and a flared portion that can be inverted so that a surface of the flared portion can be brought into contact with and attached to the stoma and/or the skin surrounding the stoma (e.g., sutured, to the stoma and/or skin). FIGS. 1a-d show an implant 100 according to one embodiment inserted and deployed in a stoma 155 while FIGS. 2a-b show an implant 200 according to another embodiment deployed in the stoma 155. FIGS. 3a-b show an implant according to yet another embodiment deployed in the stoma 155.

In these embodiments, the stoma 155 comprises a length of an intestinal lumen extending from the intestines 154 with a distal portion thereof protruding through the abdominal wall 151 and including an open end 156, as shown in FIGS. 1b-d, 2b, and 3b. It is noted that a typical stoma according to known techniques comprises a lumen that is inverted or turned out so that a distal portion comprising the end of the lumen is inverted and sewn or otherwise attached to the skin surrounding the hole in the abdomen. In the present embodiments, however, the end of the stoma 155 is not attached to the skin 153 until the introduction of an implant device such as the implant 100, 200 or 300. It is further noted that FIGS. 1b-d, 2b, and 3b show sectional views and that the lumen is tubular and the described implants comprise rounded (e.g., circular cross-sections) of varying sizes (e.g., diameters). It is further noted that the implants shown in FIGS. 1a, 1c-d, 2a-b and 3a-b are not necessarily to scale.

Implants according to the present embodiments are configured to be connected (e.g., stitched or sutured) to the stoma and/or the skin surrounding the stoma that is not expandable. The implants comprise flexible materials (e.g., Silicone) and shapes that allow a user, e.g., operating physician, to insert the implant through the stoma in a first state (e.g., an insertion state in which the implant has a first shape (undeformed)). After the implant has been inserted through the stoma, the implant is moved into a second state (e.g., a deployment state in which a flared portion of the implant is inverted) and brought into contact with the exterior surface of the stoma and the skin surrounding the stoma. The user can stitch the implant, stoma and/or skin at selected locations to complete the deployment of the implant. Accordingly, implants according to the present embodiments allow the user to control stoma size for subsequent ostomy bag placement. In some embodiments, the implant is configured for use with scaffolding for tissue integration, as will be described below in regard to FIGS. 7-10.

FIG. 1b shows a patient anatomy 150 including a stoma 155 extending through the abdominal wall 151 and through which a portion of an intestine 154 has been drawn. The abdominal wall 151 has an interior surface 152 and an exterior surface (e.g., skin 153). The lumen of the intestine is brought through the stoma 155 to protrude a distance out of the abdominal wall 151 selected for use with the implants described below, e.g., corresponding to certain dimensions of a flared portion of the implant, to be described in further detail below. The stoma 155 is generally tubular and comprises a radially inner surface 158 defining a stoma channel 157 through which waste can be passed and a radially outer surface 159, e.g., outer diameter, including a distal portion exposed to the environment exterior to the patient prior to deployment of the implant 100.

FIG. 1a shows an implant 100 comprising a body 103 and a flared portion 109 configured to be inverted during deployment of the implant 100 according to a first embodiment. The implant 100 extends from a first end 101 (proximal end) which, when the implant 100 is inserted through the stoma 155, extends into the intestine 154 to a second end 102 (distal end) which, when the implant 100 is inserted through the stoma 155, extends out of the stoma 155 facing the environment external to the patient's body. The body 103 extends from the first end 101 to a transition region 106 and the flared portion 109 extends from the transition region 106 to the second end 102. The flared portion 109 extends radially outward and away from the body 103 to define a flared shape similar to the bell of a trumpet (e.g., a shape formed by rotating a convex curve extending distally from the transition region 106 about the circumference of the transition region 106).

The implant 100 is deformable and configured to bend at the transition region 106 so that the flared portion 109 can be inverted, as shown below in FIG. 1d. That is, inversion of the implant 100 involves bending of the flared portion 109 about the transition region 106 so that the distal end of the flared portion 109 moves proximally with the radially outer surface of the flared portion 109 facing the radially outer surface of more proximal parts of the implant 100. Prior to deployment, the implant 100 is in an insertion state (undeformed) as shown in FIG. 1a, with the flared portion 109 projecting radially outward relative to the transition region 106.

The body 103, the transition region 106 and the flared portion 109 are described with regard to respective first surfaces and second surfaces. For example, the body 103 is substantially tubular such that the body 103 can be described by a first surface 104 that is a radially inner surface defining an interior channel 112 and a second surface 105 that is a radially outer surface configured to interface with the stoma 155. The transition region 106 defines a first surface 107 that is a radially inner surface (adjacent to the first surface 104 of the body 103) when the implant 100 is in the insertion state but which transitions to a radially outer surface (facing away from a center of the channel 112) when the implant 100 is in a deployment state (i.e., when the implant 100 is inverted), as shown in FIG. 1d.

A second surface 108 of the transition region 106 forms a radially outer surface during insertion and becomes a radially inwardly facing inner surface contacting the end 156 of the stoma 155 when the implant 100 is inverted. Similarly, the flared portion 109 defines a first surface 110 that is a radially inner surface (adjacent to the first surface 107 of the transition region 106) when the implant 100 is in the insertion state and becomes a radially outer surface when the implant 100 is inverted. A second surface 111 of the flared portion 109 is an outer surface during insertion and at least a portion of the second surface 111 becomes a radially inwardly facing surface contacting the outer surface 159 of the stoma 155 when the implant 100 is inverted while a distal end of the second surface 111 lays against the skin when the implant 100 is in the deployment state.

In some embodiments, the body 103, transition region 106 and flared portion 109 have substantially similar wall thicknesses. In other embodiments, the body 103 comprises a wall thickness greater than the transition region 106 and/or the flared portion 109. The body 103 is generally not designed for deformation (e.g., is not configured to bend to a degree similar to the transition region 106 and the flared portion 109) and has a radial strength sufficient to maintain its shape when implanted within the stoma 155 (i.e., to resist radially inward collapse due to pressures applied thereto by surrounding tissues). The transition region 106 and/or the flared portion 109 may, in certain embodiments, have a thinner wall to facilitate the bending necessary to transition the implant 100 from the insertion state into the deployment state. In further embodiments, such as the implant 300 described in FIGS. 3a-b, the flared portion has a second surface shaped to fit snugly into a space defined by the outer surface 159 of the stoma 155 and a portion of skin 153 adjacent to the stoma 155.

FIG. 1c shows the implant 100 of FIG. 1b inserted into the stoma 155 in the insertion state. The implant 100 (e.g., the body 103), is inserted to a desired depth in the stoma channel 157 such that the second surface 105 of the body 103 is in full contact with the inner surface 158 of the stoma 155 and the transition region 106 is adjacent to the end 156 of the stoma channel exterior to the abdominal wall 151. When the implant 100 is positioned within the stoma 155 as desired, the implant 100 is transitioned into the deployment state by pushing proximally (i.e., toward the patient's skin 153) on opposing sides of the flared portion 109 adjacent to the second end 102 of the implant in the proximal direction.

FIG. 1d shows the implant 100 of FIG. 1b in the deployment state with the flared portion 109 of the implant 100 inverted. In this example, a proximal force on opposing sides of the flared portion 109 adjacent to the second end 102 of the implant bends the transition region 106 until the second surface 108 of the transition region 106 wraps around the end 156 of the stoma 155 so that the second surface 111 of the flared portion 109 is brought into contact with the outer surface 159 of the stoma 155. The flared portion 109 of this embodiment has a size sufficient so that, when the flared portion 109 is inverted, the second end 102 of the implant 100 is brought into contact with a portion of the skin 153 adjacent to and surrounding the stoma 155.

The implant 100 can then be attached (e.g., sutured) to the stoma 155 and/or the skin 153 of the abdominal wall 151. According to an exemplary method, a first stitch 113 attaches the body 103, the stoma 155, and the flared portion 109 to one another (e.g., at a location on the stoma 155 near the end 156 of the lumen where the second surface 105 of the body 103 contacts the inner surface 158 of the stoma 155 and the second surface 111 of the flared portion 109 contacts the outer surface 159 of the stoma 155). A second stitch 114 then attaches the second end 102 of the implant 100 and/or part of the flared portion 109 to the skin 153 of the abdominal wall 151 (e.g., at a location on the flared portion 109 that was brought into contact with the skin 153).

Thus, the implant 100 can be stitched to the stoma 155 and the abdominal wall 151 fixing the implant 100 in a desired shape during the lifetime of its use and, as a result, permitting the implant 100 to maintain a consistent shape of the stoma 155 (e.g., diameter) that does not expand or contract over time.

In other embodiments, the principles described above for the implant 100 may be applied for implants having different shapes. In one example, the implant comprises a second flared portion that, when inverted, is stitched to the stoma 155 while the first flared portion is brought around the second flared portion and stitched to the skin 153. In another example, the flared portion comprises a varying thickness and an angled second surface shaped to fit snugly into the area adjacent to the outer surface 159 of the stoma 155 and onto the skin 153 adjacent to the stoma 155 when the flared portion is inverted.

FIG. 2a shows an implant 200 according to another embodiment. The implant 200 comprises a body 203, a first flared portion 209 and a second flared portion 212 configured to be inverted during deployment of the implant 200. The implant 200 extends from a first side 201 (e.g., a proximal side) which, when the implant 200 is inserted to a desired position within the stoma 155, faces the intestine 154 to a second side 202 (e.g., a distal side) which, when the implant 200 has been inserted into the stoma 155 as desired, remains exterior to the stoma 155 in a manner similar to the implant 100 of FIG. 1a. The body 203 extends from the first side 201 to a transition region 206 and the first flared portion 209 extends from the transition region 206 to the second side 202.

In this embodiment, the second flared portion 212 extends from a first end at the transition region 206 to a second end 213 and comprises a smaller flare than the first flared portion 209 (e.g., the second flared portion has a diameter at its second end 213 that is smaller than a diameter of the second side 202 of the implant 200). The implant 200 is configured to be deformed in a manner similar to that described above for the implant 100 and is configured to bend at the transition region 206 so that the first flared portion 209 and the second flared portion 212 are inverted, as shown below in FIG. 2b. Prior to deployment, the implant 200 is in an insertion state as shown in FIG. 2a with an undeformed first flared portion 209 and an undeformed second flared portion 212.

The body 203 of this embodiment is substantially tubular and defines a first surface 204 that is a radially inner surface defining an interior channel 216 and a second surface 205 that is a radially outer surface configured to interface with the stoma 155. The transition region 206 defines a first surface 207 that is a radially inner surface (adjacent to the first surface 204 of the body 203) when the implant 200 is in the insertion state and which becomes a radially outward facing surface when the implant 200 is inverted and the implant is in a deployment state, as shown in FIG. 2b.

A second surface 208 of the transition region 206 is a radially outward facing surface when the implant 200 is in the insertion state and becomes a radially inward facing surface contacting the end 156 of the stoma 155 when the implant 200 is inverted into the deployment state. The first flared portion 209 defines a first surface 210 that is a radially inward facing surface (adjacent to the first surface 207 of the transition region 206) when the implant 200 is in the insertion state and becomes a radially outward facing surface when the implant 200 is inverted into the deployment state.

A second surface 211 of the first flared portion 209 is a radially outward facing surface when the implant 200 is in the insertion state. When deformed into the deployment state, a portion of the second surface 211 becomes a radially inward facing surface facing the body 203 while a distal end of the second surface 211 extends along a surface of the skin 153 (e.g., flattened against the skin extending away from the stoma 155. The second flared portion 212 defines a first surface 214 that is a radially inward facing surface (facing toward the transition region 206 and/or the second surface 211 of the first flared portion 209) when the implant 200 is in the insertion state and becomes a radially outward facing surface when the implant 200 is inverted into the deployment state. A second surface 215 of the second flared portion 209 is a a radially outward facing surface when the implant 200 is in the insertion state and becomes a radially inward facing surface brought into contact with the outer surface 159 of the stoma 155 when the implant 200 is inverted into the deployment state.

Similarly to the implant 100 of FIG. 1a, the body 203, the transition region 206, the first flared portion 209 and the second flared portion 212 of this embodiment have wall thicknesses substantially similar to one another. Alternatively, the wall thicknesses of any of the body 203, the transition region 206, the first flared portion 209 and the second flared portion 212 may vary relative to one another and may vary along their lengths (e.g., in a proximal to distal direction) to facilitate the implantation of the body 203 in the stoma 155 and/or the bending necessary to transition the implant 200 into the deployment state.

FIG. 2b shows the implant 200 of FIG. 2a in a deployment state with the first flared portion 209 and the second flared portion 212 of the implant 200 inverted. The implant 200, e.g., the body 203 thereof, is inserted to a desired depth in the stoma channel 157 such that the second surface 205 of the body 203 is in full contact with the inner surface 158 of the stoma 155 and the transition region 206 (and the first end of the second flared portion 212) is adjacent to the end 156 of the lumen exterior to the abdominal wall 151.

In this example, a proximally directed force on radially outer (e.g., opposing sides) of the second flared portion 212 adjacent to the second end 213 bend the transition region 206 so that the second surface 208 of the transition region 206 warps around the end 156 of the stoma 155 with the second surface 215 of the second flared portion 212 being brought into contact with the outer surface 159 of the stoma 155. The second flared portion 212 of this embodiment preferably has a size selected to be sufficiently small so that the second end 213 of the second flared portion 212 does not reach the skin 153 when the implant 200 is inserted as desired into the stoma 155 and the implant 200 is deformed into the deployment state.

Prior to inverting the first flared portion 209, the second flared portion 212 and the body 203 of the implant 200 are attached (e.g., sutured) to the stoma 155. According to an exemplary method, a first stitch 217 attaches the body 203 and the stoma 155 (and optionally the skin 153) to one another at a location on the stoma 155 adjacent to the skin 153. A second stitch 218 then attaches the body 203, the stoma 255, and the second flared portion 212 to one another (e.g., at a location on the stoma 155 where the second surface 205 of the body 203 contacts the inner surface 158 of the stoma 155 and the second surface 215 of the second flared portion 209 contacts the outer surface 159 of the stoma 155).

After the second flared portion 212 has been secured to the stoma 155, a proximally directed force is applied to radially outer portions of (e.g., opposing sides) of the first flared portion 209 adjacent to the second side 202 of the implant 200 to invert the first flared portion 209. The first flared portion 209 of this embodiment is preferably selected to have a size sufficient so that, when inverted, the second side 202 of the implant 200 is brought into contact with the skin 153 surrounding the stoma 155. A third stitch 219 can then attach the second side 202 of the implant 200 and/or part of the flared portion 209 to the abdominal wall 151, e.g., at a location on the flared portion 209 that was brought into contact with the skin 153.

Thus, the implant 200 can be secured to the stoma 155 and the abdominal wall 151 in a position and orientation selected so that the implant 200 retains a desired shape during its lifetime of use and, as a result, the stoma 155 maintains a consistent shape (e.g., diameter) and does not expand or contract over time. As would be understood by those skilled in the art, the two flared portions of the implant 200 may provide additional anchoring locations for suturing to better secure the implant 200 in place.

FIG. 3a shows an implant 300 according to a further embodiment. The implant 300 comprises a body 303 (e.g., a substantially tubular body) and a flared portion 309 shaped to fit snugly into an area adjacent to the stoma 155 and the skin 153 surrounding the stoma 155 when the flared portion 309 is inverted. The implant 300 comprises certain features substantially similar to those of the implant 100 of FIG. 1a. In particular, a body 303 of the implant 300 extends from a first side 301 of the implant 300 to a transition region 306 and a flared portion 309 extends from the transition region 306 to a second end 302 of the implant 300. The implant 300 is configured to bend at the transition region 306 so that the flared portion 309 is inverted, as shown below in FIG. 3b.

Similar to the implant 100 of FIG. 1a, the body 303, the transition region 306 and the flared portion 309 of the implant 300 define first surfaces and second surfaces, with the body 303 comprising a first surface 304 that is a radially inward facing surface defining an interior channel 314 and a second surface 305 that is a radially outward facing surface configured to interface with a radially inner surface of the stoma 155 and the transition region 306 defines a first surface 307 that is a radially inward facing surface when the implant 300 is in the insertion state and becomes a radially outward facing surface when the implant 300 is in the deployment state, as shown in FIG. 3b. A second surface of the transition region 306 is a radially outward facing surface when the implant 300 is in the insertion state and becomes a radially inward facing surface contacting the end 156 of the stoma 155 when the implant 300 is inverted into the deployment state.

The flared portion 309 comprises a first surface 310 that is a radially inward facing surface (adjacent to the first surface 307 of the transition region 306) when the implant 300 is in the insertion state and becomes a radially outward facing surface when the implant 300 is inverted into the deployment state and a second surface 311 that is a radially outward facing surface when the implant 300 is in the insertion state and a portion of which becomes a radially inward facing surface contacting the outer surface 159 of the stoma 155 when the implant 300 is inverted into the deployment state while a distal-most portion of the flared portion 309, when the implant 300 is inverted to the deployment state lays against a surface of the skin 153 surrounding the stoma 155 in a manner similar to the implant 100 of FIG. 1a. In this embodiment, the second surface 311 of the flared portion 309 is shaped so that the second surface 311 can be brought into full contact with the stoma 155 and the skin 153 with little or no space between the second surface 311 and patient anatomy 150.

The second surface 311 of the flared portion 309 comprises a first part 312 extending at a shallow angle from the transition region 306 at a first end to a relatively sharply defined angle that is substantially transverse to a second part 313 that extends proximally from the angle to the second end 302 of the implant 300. In this example, the flared portion 309 comprises a wall substantially thicker than the body 303. This thickened wall is configured to enhance the strength of the implant 300 permitting the implant 300 to be secured to the patient anatomy 150 as desired with a reduced number of stitches as compared to the implants 100 and 200. That is, as would be understood by those skilled in the art, because the shape of the second surface of the 311 is selected to conform (when the implant 300 has been inverted, to a shape of the space at which the stoma 155 meets the skin 153, less sutures will be required to achieve an effective seal around the stoma 155.

The first surface 310 of the flared portion 309 of this embodiment is shaped in a manner similar to the second surface 311, e.g., having a first part extending to an angled corner from which a second part extends, as shown in FIG. 3b below. Alternatively, the first surface 310 may follow a continuous curve similar to that of the implant 100. The first surface 310 can be shaped to facilitate the deployment of the implant 300 (e.g., having a region (angled region) suitable for a physician to press down (toward the skin) with one or more fingers to provide a force keeping the flared portion snugly fit into the space around the stoma 155 and the skin 153) as will be described in further detail below.

FIG. 3b shows the implant 300 of FIG. 3a in a deployment state with the flared portion 309 of the implant 300 inverted. The implant 300, e.g., the body 303 thereof, is inserted to a depth in the stoma channel 157 such that the second surface 305 of the body 303 is in full contact with the inner surface 158 of the stoma 155 and the transition region 306 is adjacent to the end 156 of the lumen exterior to the abdominal wall 151. In this example, a proximal force applied to opposing sides of the flared portion 309 bends the transition region 306 bringing the second surface 308 of the transition region 306 around the end 156 of the stoma 155.

This brings the first part 312 of the second surface 311 of the flared portion 309 into contact with the outer surface 159 of the stoma 155. The stoma 155 can be pulled distally through the abdominal wall 151 a distance matching the length of the first part 312 of the second surface 311 of the flared portion 309 so that the angle of the second surface 311 is brought to and pressed into the location at which the outer surface 159 of the stoma 155 meets the skin 163. From here, the second part 313 of the second surface 311 extends along the skin 163 away from the stoma 155.

The implant 300 can then be stitched to the stoma 155 and the abdominal wall 151 in a manner similar to that described above in regard to the implants 100, 200. Alternatively, a first stitch 315 can attach the body 303 and the stoma 155 to one another (and optionally the skin 153) at a location on the stoma 155 adjacent to the skin 153. The first stitch 315 can be applied prior to inverting the implant 300. The implant 300 may then be inverted into the deployment state as described above and a second stitch 316 can then attach the second end 302 of the implant 300 and/or part of the flared portion 309 to the abdominal wall 151.

Thus, the implant 300 can be stitched to the stoma 155 and abdominal wall 151 so that the implant 300 is fixed in a shape that remains substantially unchanged during its lifetime of use and, as a result, the stoma 155 is maintained in a substantially consistent size and shape (e.g., diameter) without significantly expanding or contracting over time. Relative to the implant 100 described above in FIG. 1a and the implant 200 described above in FIG. 2a, the implant 300 may provide additional stability by filling the space between the implant 300 and the patient anatomy 150 more completely and making it easier to achieve a seal around the stoma 155. As indicated above, the implant 300 may also be sufficiently sealed around the stoma 155 while using fewer stiches than required for the implant 200 of FIG. 2a.

In other embodiments, an implant comprises a volume including at least a portion, e.g., a body portion, that has a ring-like shape that forms a surface external to the abdominal wall around which an inverted intestinal lumen can be wrapped and secured to the implant and/or the skin to form a stoma. FIG. 4 shows an implant 400 according to an embodiment configured to provide a surface for the formation of a stoma. FIG. 5 shows an implant 500 including a body configured to provide a surface to form a stoma and a skin abutting surface configured to form a skin barrier external to the abdominal wall according to another embodiment.

FIG. 6 shows an implant including a body configured to provide a surface to form a stoma and a skin abutting surface configured to form a barrier between the surface of the target organ (e.g., an intestine) to be drawn through the abdominal wall to for the stoma. The implant of FIG. 6 includes a proximal portion forming an internal barrier between the intestine and the inner surface of the abdominal wall and an external barrier between the skin surrounding the stoma and the intestinal tissue according to still another embodiment. In these embodiments, the stoma is formed by inverting the lumen of the implant and stitching its end to either or both of the skin surrounding the stoma and/or the implant. In other words, the stomas formed with these embodiments more closely resembles a typical stoma, e.g., relative to the implants 100-300 described above, and, as a result, current ostomy bags and/or a wider range of ostomy-related devices can be used with the implants 400-600.

It is further noted that FIGS. 4-6 show sectional views and that the lumens of these implants are generally tubular and the described implants comprise shapes having consistent cross-sections each forming a three-dimensional ring. The implants are configured to be placed on the patient anatomy so that the lumen of the target organ can be brought through a center of the stoma, inverted around the surface of the implant and stitched to the skin and/or implant. It is further noted that the implants shown in FIGS. 4-6 are not necessarily to scale.

In the present embodiments, the implant or a body thereof may be radially expandable. In various embodiments, the implant or body thereof can expand in a first radial direction, e.g., inward, a second radial direction, e.g., outward, or in either direction depending on the desired result. The implant may be positioned as desired relative to the stoma (e.g., inserted into the stoma in a compressed state and allowed to expand after placement of the implant in the patient anatomy. In various embodiments, the implant may or may not have sutures or other mechanistic ties to the patient anatomy.

In some embodiments, the user can stitch the implant, stoma and/or skin to one another at

selected locations to complete the deployment of the implant. In other embodiments, the implant may sit within the patient anatomy. Accordingly, the implants according to the present embodiments allow user control the stoma size for subsequent bag placement onto the skin. In some embodiments, the implant can be used with scaffolding for tissue integration, as will be described below with regard to FIGS. 7-10.

FIGS. 4-6 show a patient anatomy 450 including a stoma 455 formed by extending a portion of the intestines 454 through an opening in the abdominal wall 451 with the portion of the intestines 454 protruding through the abdominal wall 451 being inverted. The abdominal wall 451 has an interior surface 452 and an exterior surface (e.g., skin 453). During deployment of the implant 400, a portion of the intestines 454 is brought through a hole in the abdominal wall 451 to a length selected for use with the implants described below, e.g., corresponding to certain dimensions of the implant, to be described in further detail below.

A first part of the stoma 455 comprises a portion of the intestinal lumen extending through the abdominal wall 451, e.g., in non-inverted form, and a second part of the stoma 455 comprises the inverted distal portion of the intestinal lumen and an end 456 of the lumen. The first part of the stoma 455 is substantially tubular and comprises an inner surface 458 defining a stoma channel 457 through which waste can pass and an outer surface 459 in contact with a radially inner surface of the hole in the abdominal wall 451 and, when the implant is deployed, in contact with a surface or surfaces of the implant.

The second part of the stoma 455 is formed by a curved portion of the intestinal wall inverted so that a first surface 460 (that previously formed an inner surface at the end of the lumen) forms the outer surface of the second part (e.g., exposed to the environment) and a second surface 461 (that previously formed an outer surface at the end of the lumen) forms the inner surface of the second part that is brought into contact with a surface or surfaces of the implant. The end 456 of the stoma 455 is then secured to the implant and/or skin 453.

FIG. 4 shows an implant 400 configured to provide a surface for supporting tissue to form a stoma 455. The implant 400 of FIG. 4 is shown deployed and attached to the patient anatomy as 450 described above. The implant 400 comprises a first surface 401 (e.g., an inner surface), a second surface 402 (e.g., a top surface), a third surface 403 (e.g., an outer surface), and a fourth surface 404 (e.g., a bottom surface). It should be understood that the implant 400 is described with regard to four surfaces (and/or two-dimensional sides of the cross section of the implant 400) for ease of description and comprises a three-dimensional shape that is ring-like, e.g., comprises a cross-sectional shape of the implant 400 revolved about a circle or other curved closed shape. It should be understood that the cross-sectional geometry, as shown in FIG. 4, is described with regard to intersection points between the surfaces 401-404 of the implant 400, however, this is only for ease of description and the geometry of the cross-section of the implant 400 can comprise a combination of angles (either sharply defined or slightly rounded) and curves.

In this example, the first surface 401 and the fourth surface 404 intersect at a relatively sharply defined orthogonal angle that is shaped to fit snugly into the area adjacent to the second surface 402 of the first (non-inverted) part of the stoma 455 and the skin 453 surrounding the hole in the abdomen. Those skilled in the art will ascertain that this intersection point between the first and fourth surfaces 401, 404 can be slightly rounded for atraumatic deployment and use of the implant 400. The first surface 401 extends away from the fourth surface 404 and transitions into the second surface 402.

Those skilled in the art will ascertain that this intersection point between the first and second surfaces 401, 402 can, in other embodiments, be a more sharply defined angle relative to that shown in FIG. 4 for providing a corner surface over which the lumen can be inverted. The second surface 402 curves and transitions into the third surface 403. Those skilled in the art will ascertain that this rounded top surface is not required. In other embodiments, the second surface of the implant could extend away from the first surface and toward the bottom surface to form a substantially triangular shape.

In this example, the third surface 403 extends back toward the fourth surface 404 and can further extend away from the first surface 401 to provide an angled surface, relative to the skin 453, over which the lumen can be drawn back toward the skin 453 after inversion. The fourth surface 404 extends from an intersection point with the third surface 403 to the intersection point with the first surface 401. Those skilled in the art will ascertain that this intersection point between the third and fourth surfaces 403, 404 can be sharply defined or substantially rounded. Those skilled in the art will further ascertain that these surfaces 401-404 and intersection points therebetween are loosely defined and that the cross section of the implant 400 can comprise a substantially rounded surface with no strictly visible intersection points or can include one or more sharply defined intersection points between two of the at least three surfaces (or sides of the cross section).

To deploy the implant 400, the fourth surface 404 is placed against the skin around the hole in the abdominal wall 451 and the portion of the intestine for forming the stoma 455 is brought out of the body through the hole (or, if desired, the intestinal tissue can be brought through the hole prior to placing the implant 400). The operating physician may then check to ensure that the portion of the intestinal tissue brought through the hole is sufficient to form the stoma 455 around the implant 400. The outer surface 459 of the first part of the intestinal tissue is brought into contact with the first surface 401 of the implant and the intestinal lumen is inverted so that a second surface 461 of the second part of the lumen is brought into contact with the second and third surfaces 402, 403 and the end 456 of the intestinal tissue is brought into contact with the skin 453.

The end 456 of the intestinal tissue is then secured to the skin 453 adjacent to the intersection between the third and fourth surfaces 403, 404 of the implant 400 to form the stoma 455. In this embodiment, the end 456 of the intestinal tissue is sutured to the skin 453 via stitching 405 and is not sutured or otherwise attached to the implant 400. It should be understood that the implant 400 or implants within the scope of the present embodiment as described above may or may not be directly attached to the patient anatomy 450. The implant 400 may sit in the space formed between the first and second parts of the stoma and the skin, may be sutured in place, or may be used with other devices/implants such as, e.g., a scaffold for tissue integration similar to those described below with regard to FIGS. 7-10.

After the implant 400 is deployed it can be allowed to expand. In one embodiment, the implant 400 expands radially outward, e.g., the third surface 403 expands away from the first surface 401 (labeled “A” in FIG. 4) and the first surface 401 substantially maintains the location and diameter at which it was placed. In another embodiment, the implant 400 expands radially inward, e.g., the first surface 401 expands away from the third surface 403 (labeled “B” in FIG. 4) and the third surface 403 substantially maintains the location and diameter at which it was placed. In still another embodiment, the implant 400 expands both radially outward and radially inward (labeled “C” in FIG. 4) and some location between the first and third surfaces 401, 403 maintains the location and diameter at which it was placed.

Thus, the implant 400 can be deployed so that the implant 400, after it expands, is fixed in a substantially same shape during the lifetime of its use and, as a result, the stoma 455 maintains a consistent shape (e.g., diameter) and does not expand or contract over time.

In other embodiments, the principles described above for the implant 400 can be applied for implants having different shapes and/or additional features. In one embodiment, the implant comprises a skin barrier attached to or formed with a body that provides a surface to aid in forming the stoma. In another example, the skin barrier includes a first portion for implantation on the exterior surface (skin) of the abdominal wall, a second portion for implantation on the interior of the abdominal wall, and a bridge portion connecting and defining a gap between the first and second portions for interfacing with the tissue of the abdominal wall surrounding the hole in the abdomen.

FIG. 5 shows an implant 500 including a body 510 for providing a surface to form a stoma and a skin barrier 520 external to the abdominal wall 451 according to another embodiment. The implant 500 of FIG. 5 is shown deployed and attached to the patient anatomy 450. It is noted that certain aspects of the patient anatomy 450 are not labeled in FIG. 5 and substantially correspond to those aspects shown in FIG. 4 unless described otherwise below. The implant 500 comprises certain features similar to those of the implant 400 of FIG. 4. In particular, a shape of a body 510 of the implant 500 is similar to the shape of the implant 400 and provides a surface on which the second (inverted) part of the stoma is formed. In the present embodiment, however, the body 510 is attached to or formed with a skin barrier 520.

The body 510 comprises a first surface 501 (e.g., an inner surface), a second surface 502 (e.g., a top surface), a third surface 503 (e.g., an outer surface), and a fourth surface 504 (e.g., a bottom surface) similar to the implant 400 of FIG. 4. The fourth surface 504 of the body 510 is configured for attachment to the skin barrier 520. In this example, the skin barrier 520 has a substantially triangular cross section including a first surface 521 (e.g., an inner surface), a second surface 522 (e.g., an outer surface), and a third surface 523 (e.g., a bottom surface). The first surface 521 of the skin barrier 520 is configured to be attached to and coterminous with the fourth surface 504 of the body 510.

The body 510 is shaped similarly to the implant 400 of FIG. 4. However, in this example, the fourth surface 504 is angled distally from its intersection point with the first surface 501 to its intersection point with the third surface 503. The first surface 521 of the skin barrier 520 extends from an intersection point with the third surface 523 to an intersection point with the second surface 522. The combined angles between the third surface 523 of the skin barrier 520 and the first surface 501 of the body 510 approximates 90 degrees. The angle between the first and second surfaces 521, 522 is large and forms a top corner of a relatively flat triangle. The third surface 523 extends radially outward from its intersection point with the first surface 521 to its intersection point with the second surface 522.

Those skilled in the art will further understand that the surfaces 501-504 and the 521-523 intersection points therebetween are loosely defined and that the cross section of the implant 500 can comprise a substantially rounded exterior surface with no strictly visible intersection points or may, alternatively, include one or more sharply defined intersection points between the surfaces. It should be understood that the implant 500 comprises a three-dimensional shape that is ring-like, e.g., comprises a cross-sectional shape revolved about a circle (or other closed curve), and is described with regard to the aforementioned two-dimensional surfaces (and/or sides of the cross section of the implant 500) for ease of description.

It is further noted that the fourth surface 504 of the body 510 and the first surface 521 of the skin barrier 520 may not be strictly defined. For example, the body 510 and the skin barrier 520 may be formed together and not as separate components that are subsequently attached to one another. In this example, the body 510 and the skin barrier 520 comprise separate components. In particular, the body 510 is configured to expand after implantation while the skin barrier 520 may not have expandable qualities.

To deploy the implant 500, the third surface 523 of the skin barrier 520 is placed around the hole in the abdominal wall 451 and the tissue of the target organ (e.g., an intestine) for forming the stoma 455 is brought through the hole to extend out of the patient's body. The operating physician then checks to ensure that the portion of tissue of the target organ that is brought out through the hole is sufficient to form the stoma 455 around the implant 500. The outer surface 459 of the first part of the tissue is brought into contact with the first surface 501 of the body 510. The portion of the target organ is then inverted so that a second surface 461 of the second part of the tissue is brought into contact with the second and third surfaces 502, 503 and the end 456 of the tissue is attached to the body 510 and/or the skin barrier 520 adjacent to the intersection between the third and fourth surfaces 503, 504 of the body 510 and/or the intersection between the first and second surfaces 521, 522 of the skin barrier 520 to form the stoma 455. In this embodiment, the end 456 of the tissue of the target organ is stitched to the implant 500 with first stitching 505 in the location described above and the skin barrier 520 is stitched to the skin 453 with second stitching 506 adjacent to the intersection between the second and third surfaces 522, 523.

After the implant 500 is deployed the body 510 is allowed to expand. In one embodiment, the body 510 expands radially outward, e.g., the third surface 503 expands away from the first surface 501 (labeled “A” in FIG. 5) and the first surface 501 substantially maintains the location and diameter at which it was placed. In another embodiment, the implant 500 expands radially inward (e.g., the first surface 501 expands away from the third surface 503 (labeled “B” in FIG. 5) and the third surface 503 substantially maintains the location and diameter at which it was placed). In still another embodiment, the implant 500 expands both radially outward and radially inward (labeled “C” in FIG. 5) with a portion of the implant 500 between the first and third surfaces 501, 503 maintaining its location at which it was placed as well as its original diameter.

Thus, the implant 500 can be deployed so that the implant 500, after the body 510 has expanded, is fixed in a shape and location that remains substantially the same during its lifetime of use and, as a result, the stoma 455 maintains a consistent shape (e.g., diameter) and location and does not expand or contract over time. The skin barrier 520 further protects the skin 453 as would be understood by those skilled in the art.

FIG. 6 shows an implant 600 including a body 610 configured to provide a surface to form a stoma 455 and a skin barrier 620 both internal and external to the abdominal wall 451. The implant 600 of FIG. 6 is shown deployed and attached to the patient anatomy 450. It is noted that certain aspects of the patient anatomy 450 are not labeled in FIG. 6 and substantially correspond to those aspects shown in FIG. 5 unless described otherwise below. The implant 600 comprises certain features similar to those of the implant 500 of FIG. 5.

In particular, a shape of a body 610 of the implant 600 is substantially similar to the shape of the body 510 of the implant 500 of FIG. 5 and provides a surface on which the second (inverted) part of the stoma is formed. Additionally, a shape of a first portion 621 (e.g., external portion) of a skin barrier 620 is substantially similar to the shape of the skin barrier 520 of the implant 500 of FIG. 5. In the present embodiment, however, the skin barrier 620 further includes a second portion 623 (e.g., internal portion) configured so that, when the implant 600 is inserted, the second portion 623 contacts an interior surface 452 of the abdominal wall 451.

The body 610 comprises a cross-sectional shape and surfaces substantially similar to those described above for the body 510 of the implant 500 of FIG. 5. The bottom surface of the body 610 is configured for attachment to the skin barrier 620. In this example, the first portion 621 of the skin barrier 620 has a substantially triangular cross section substantially similar to that of the skin barrier 520 of the implant 500 of FIG. 5. A bridge portion 622 connects the first portion 621 and the second portion 623. The second portion 623 includes a first part extending proximally away from the bridge and defining a first surface 624 contacting the outer surface of the lumen and a second part extending radially away from the first surface 624 and defining a second surface 625 contacting the interior surface 452 of the abdominal wall 451.

Those skilled in the art will further understand that the shape of the second portion 623 of the implant 600 may vary. It is further noted that the boundary between the body 610 and the skin barrier 620 need not be strictly defined, e.g., the body 610 and the skin barrier 620 may be formed together and not as separate components that are subsequently attached to one another. In this example, the body 610 and the skin barrier 620 comprise separate components and, similar to the implant 500 of FIG. 5, the body 610 of the implant 600 may be configured to expand after implantation while the skin barrier 620 may not have expandable qualities.

The implant 600 can be deployed in a manner similar to that described above for the implant 500 of FIG. 5. For example, the implant 600 of FIG. 6 is placed in the patient anatomy 450 prior to bringing a portion of the target organ through the implant 600. The portion of the target organ is then inverted and secured to the implant 600 in a manner substantially similar to that described above for the implant 500 of FIG. 5, including, for example, similar locations for the stitching. After the implant 600 is deployed the body 610 may be allowed to expand, e.g., radially outward (labeled “A” in FIG. 6), radially inward (labeled “B” in FIG. 6) or both radially outward and radially inward (labeled “C” in FIG. 6).

Thus, the implant 600 can be deployed so that the implant 600, after the body 610 expands, is fixed in a substantially same shape and location during its lifetime of use and, as a result, the stoma 455 maintains a consistent shape (e.g., diameter) and does not expand or contract over time. The skin barrier 620 further protects the skin 453 and the interior surface 452 of the abdominal wall 451.

It is noted that the preceding embodiments are generally compatible with tissue integration devices as described below with regard to FIGS. 7-10. It is further noted that the preceding embodiments can be compatible with current ostomy bags as well as alternative connection mechanisms with ostomy bags or waste encapsulation connectivity via built in mechanisms as described below with regard to FIGS. 11-13.

In other embodiments, an implant comprises a scaffold for a stoma that can be either partially internal, fully internal or fully external to the abdominal wall. The implant can comprise a ring-like shape that forms a surface to which tissue of a target organ (e.g., an intestine) can be secured to form a stoma. In some embodiments, a cross-section of the implant comprises an L-shape including a first surface configured for attachment to the tissue of the target organ and a second surface for attaching to the abdominal wall. In some embodiments, the implant provides a distal region for attaching an ostomy bag.

FIG. 7 shows an implant 700 providing a scaffold configured to be positioned, when the implant 700 is implanted as desired, to external partially through the abdominal wall for supporting a stoma according to one embodiment. FIG. 8 shows an implant 800 providing a scaffold configured to be placed fully external to the abdominal wall for supporting a stoma according to another embodiment. FIG. 9 shows an implant 900 for supporting a stoma according to another embodiment. The implant 900 provides a scaffold that is, when implanted as desired, entirely internal to the abdominal wall. FIG. 10 shows an implant 1000 according to a further embodiment that provides a scaffold that is implanted internal to the abdominal wall and internal to the lumen of the target organ for supporting a stoma.

FIG. 11 shows an ostomy bag 1100 for use with the implants 700, 800 of FIGS. 7-8 In these embodiments, the stoma 755 comprises a length of an intestinal lumen extending from the intestines and including an open end 756 of the lumen, as shown in FIGS. 7-10. In the present examples, the end 756 of the stoma 755 is not attached (directly or indirectly) to the abdominal wall 751 an implant device, e.g., implant 700, 800, 900 or 1000 has been positioned as desired. It is further noted that FIGS. 7-10 show sectional views of the implant. The lumen is tubular and the described implants comprise a cross-sectional shape revolved around a circle (or other continuous curve) to define a three-dimensional ring-like shape. It is further noted that the implants shown in FIGS. 7-10 are not necessarily to scale.

It is noted that existing ostomy bags are often attached directly to the skin with an adhesive. The adhesive material, along with leaks from the bag coming in contact with the skin can cause acute and chronic irritation. The present implants (or scaffolds) for the stoma solve this problem by surrounding and supporting the stoma and providing a surface on which the bag can be mounted. The scaffolds can be made of materials that allow for tissue integration and ingrowth, including collagen, alginate, silk, PLA, PLGA, and PCL. It is further noted that some of the presently described scaffolds may be compatible with some of the implants of the previous embodiments.

FIG. 7 shows an implant 700 providing a scaffold that is configured for implantation so that the implant 700 extends partially external and partially internal to the abdominal wall. The implant 700 of FIG. 7 is shown deployed and attached to patient anatomy 750 in a desired position. The patient anatomy 750 includes a stoma 755 formed via a lumen of a portion of an intestine extending out of the patient's body through the abdominal wall 751. The abdominal wall 751 has an interior surface 752 and an exterior surface (e.g., skin 753).

The intestinal tissue forming the stoma 755 is brought through a hole in the abdominal wall 751 to protrude a distance out of the abdominal wall 751 selected for use with the implant 700, e.g., corresponding to a distance the implant 700 protrudes from the abdominal wall 751 when placed in the patient anatomy 750, to be described in further detail below. The intestinal lumen is tubular and comprises an inner surface 758 defining a stoma channel 757 through which waste can be passed and an outer surface 759, e.g., outer diameter. The lumen has the end 756. It is noted that the nomenclature of the patient anatomy 750 of FIG. 7 is also used to describe the implants of FIGS. 8-10, however, in FIGS. 9-10, the lumen forming the stoma 755 remains internal to the abdominal wall 751.

The implant 700 comprises a cross-sectional shape similar to an L including a first portion 701 extending in a first direction (e.g., parallel to a longitudinal axis of the stoma in a proximal-distal direction), a second portion 702 extending in a second direction selected to match a surface of the skin (e.g., orthogonal to the first direction (e.g., radially outward from the stoma)), and a corner portion 703 defining the angle between the first and second portions 701, 702.

In this example, the first portion 701 extends distally from the corner portion 703 and forms a first surface 704 (e.g., an internal surface of the implant 700 for contacting the outer surface 759 of the lumen) and a second surface 705 (e.g., an outer surface that can include a region 706 for attaching an ostomy bag) to be described in further detail below. The second portion 702 extends radially outward from the corner portion 703 and forms a third surface 707 (e.g., a top surface for contacting the interior surface 752 of the abdominal wall 751), and a fourth surface 708 (e.g., a bottom surface facing the GI tract). In this example, the first portion 702 has a length sufficient to pass through and protrude a distance past the abdominal wall 751 from the corner portion 703 that is internal to the abdominal wall 751.

To deploy the implant 700, the implant 700 is positioned through the hole in the abdominal wall 751 so that the second portion 702 is internal to the abdominal wall 751 and the third surface 707 contacts the interior surface 752 of the abdominal wall 751. The intestinal tissue is then brought through the center channel of the implant 700 so that the outer surface 759 of the lumen contacts the first surface 704 of the first portion 701 of the implant 700. The intestinal tissue is then stitched to the first portion 701 at multiple locations along its length. The second portion 702 can be stitched to the abdominal wall 751 at multiple locations along its length.

Thus, the partially internal implant 700 of FIG. 7 is mounted between the abdominal wall 751 and the GI tract and surrounds the stoma 755 on its exterior. An ostomy bag can then be attached to the region 706 on the second surface 705 of the first portion 701 of the implant. Thus, the bag is mounted directly to the implant 700 and not to the skin, thereby preventing irritation due to adhesive and minimizing leaks encountering the skin.

FIG. 8 shows an implant 800 providing a scaffold fully external to the abdominal wall 751 configured to support a stoma 755 according to another embodiment. The implant 800 of FIG. 8 is shown deployed and attached to the patient anatomy 750. The patient anatomy 750 is substantially similar to that shown in FIG. 7. Similar to the implant 700, the implant 800 comprises a cross-sectional shape similar to an L including a first portion 801 extending in a first direction, a second portion 802 extending in a second direction orthogonal to the first direction (radially outward), and a corner portion 803 defining the angle between the first and second portions 801, 802.

The first portion 801 extends distally from the corner portion 803 and forms a first surface 804, e.g., an internal surface configured to contact the outer surface 759 of the intestinal tissue, and a second surface 805, e.g., an external surface. The second portion 802 extends radially outward from the corner portion 803 and forms a third surface 806 (e.g., a top surface that can include a region 807 for attaching an ostomy bag) to be described in further detail below. The second portion 802 has a fourth surface 808 for contacting the skin 753 of the abdominal wall 751. In this example, the first portion 801 has a shortened length relative to the first portion 701 of the implant 700 of FIG. 7 as this length is fully external to the abdominal wall 751.

To deploy the implant 800, the implant 800 is placed on the skin 753 surrounding the hole in the abdominal wall 751 so that the second portion 802 is external to the abdominal wall 751 and the fourth surface 808 contacts the skin 753. The intestinal tissue is then brought through the center channel of the implant 800 so that the outer surface 759 of the lumen contacts the first surface 804 of the first portion 801 of the implant 800. The intestinal tissue is then secured to the first portion 801 (e.g., via suturing at multiple locations along its length). The second portion 802 is then secured to the abdominal wall 751 (e.g., via suturing at multiple locations along its length).

An ostomy bag can then be attached to the region 807 on the third surface 806 of the second portion 802. Thus, the bag is mounted directly to the implant 800 and not to the skin, thereby preventing irritation due to adhesive and minimizing leaks encountering the skin.

In the embodiments described above with regard to FIGS. 7-8 the ostomy bag is attached to the scaffold itself so that the skin does not come in contact with potentially irritating adhesives from the bag. The partially internal scaffold of FIG. 7 will require an altered bag adhesive design for the desired attachment site.

In further embodiments, the scaffold may also be attached such that there is little or no external protrusion of the scaffold. This prevents output/skin contact and therefore potential irritation. It also provides direct scaffold to bag connection. The portion of the scaffold extending internally may be varied in material stiffness or cross section, to maintain patency.

FIG. 9 shows an implant 900 providing a scaffold internal to the abdominal wall 751 for supporting a stoma 755 according to still another embodiment. The implant 900 of FIG. 9 is shown deployed and attached to patient anatomy 750 in a desired position and orientation. The patient anatomy 750 is substantially similar to that shown in FIGS. 7-8 except where otherwise indicated. In this example, the tissue of the target organ is not brought through the hole in the abdominal wall 751.

Similarly to the implants 700, 800, the implant 900 comprises a cross-sectional shape similar to an L including a first portion 901 extending in a first direction, a second portion 902 extending in a second direction orthogonal to the first direction, and a corner portion 903 defining the angle between the first and second portions 901, 902. The first portion 901 extends proximally from the corner portion 903 and forms a first surface 904, e.g., an internal surface configured to contact the outer surface 759 of the lumen, and a second surface 905 facing the GI tract. The second portion 902 extends radially outward from the corner portion 903 and forms a third surface 906, e.g., a top surface configured to contact the interior surface 752 of the abdominal wall 751. The second portion 902 has a fourth surface 907 configured to face the GI tract when the implant is positioned as desired.

In this example, the implant 900 further has a distal portion 908 extending distally and radially internally off the corner portion 903. The distal portion 908 is configured to interface with the end 756 of the tissue of the target organ and the hole in the abdominal wall 751 and to support a bag attachment region 909.

To deploy the implant 900, the implant 900 is inserted through the hole in the abdominal wall 751 with the distal portion 908 contacting the tissue surrounding the hole in the abdominal wall 751 (i.e., forming the radially inner surface of the hole in the abdominal wall 751), the first portion 901 extending proximally into the patient's body from the second portion 902. The second portion 902 is configured to remain internal to the abdominal wall 751 with the third surface 906 contacting the inner surface of the abdominal wall 751. The tissue of the target organ is drawn out through the center channel of the implant 900 so that the outer surface 759 of the lumen contacts the first surface 904 of the first portion 901 of the implant 900 and the end 756 contacts the distal portion 908 at its radially inward part. The tissue of the target organ is then secured to the first portion 901 (e.g., via suturing at multiple locations along its length). The second portion 902 can then be secured to the abdominal wall 751 (e.g., via suturing at multiple locations along its length).

An ostomy bag can be attached to the region 909 on the distal portion 908. Thus, the bag is mounted directly to the implant 900 and not to the skin, thereby preventing irritation due to adhesive and minimizing leaks encountering the skin. The region 909 can be completely internal to the skin 753 or can protrude a small distance out.

FIG. 10 shows an implant 1000 providing a scaffold internal to the abdominal wall 751 and internal to the lumen for supporting a stoma 755 according to still another embodiment. The implant 1000 of FIG. 10 is shown deployed and attached to patient anatomy 750 in a desired position and orientation. The patient anatomy 750 is substantially similar to that shown in FIG. 9 except where otherwise indicated. In this example, the tissue of the target organ is not brought through the hole in the abdominal wall 751 and a proximal portion of the implant 1000 remains within the tissue of the target organ. The implant 1000 of this embodiment has a cross-sectional shape that is relatively straight and includes a first portion 1001 extending in a first direction and a second portion 1002 extending off the first portion 1001 in the same direction. The first and second portions 1001, 1002 extend proximally from a distal end 1003 and form a first surface 1004, e.g., an external surface configured to contact the radially inner surface 758 of the tissue of the target organ. The distal end 1003 is configured to interface with the hole in the abdominal wall 751 and to support an ostomy bag attachment region 1005.

To deploy the implant 1000, the implant 1000 is inserted through the hole in the abdominal wall 751 so that the distal end 1003 thereof contacts the tissue surrounding the hole in the abdominal wall 751 and the first and second portions 1001, 1002 extend proximally into the body. The tissue of the target organ is placed around the first surface 1004 so that the inner surface 758 of the tissue of the target organ contacts the first surface 1004 and the end 756 of the tissue contacts the interior surface 752 of the abdominal wall 751. The tissue of the target organ is then secured to the first surface 1004 (e.g., via suturing at multiple locations along its length) and/or the end 756 can be secured to the interior surface 752 of the abdominal wall.

An ostomy bag can be attached to the region 1005 on the distal end 1003. Thus, the bag is mounted directly to the implant 1000 and not to the skin, thereby preventing irritation due to adhesive and minimizing leaks encountering the skin. The region 1005 can be completely internal to the skin 753 or can protrude a small distance out.

FIG. 11 shows an ostomy bag 1100 including a region 1101 configured to interface with an implant according to one or more of the previous embodiments. For those implants having a distal protrusion, e.g., implants 700, 800, 900 or 1000, the region 1101 can be sized and shaped to match the dimensions of the implant for a bag attachment region. The region 1101 of the ostomy bag 1100 can have an adhesive for attaching to the implant.

Currently, most ostomy devices are coupled to the skin/stoma via an adhesive. These adhesives/materials are cut manually by the user using scissors after measuring the size of the stoma using gauges (as the stoma can change size over time). Because of this, the adhesive cut can be rough and unreliable, hence the high frequency of leaking. One solution to this issue is adding a design of concentric rings with breathing holes which will be able to adhere to the skin around the stoma.

FIG. 12 shows an adhesive material 1200 including a number of rings 1201 (e.g., concentric rings) composed of perforations in the material 1200. The rings 1201 are concentric and each define a different size circle. In this embodiment, the rings 1201 are formed of close perforations which would make it easier for a user to create an attachment to the skin of adequate size simply by tearing along the adequate perforations. These perforations have a size selected to prevent liquid and solid waste from passing therethrough but will allow overall material breathability of the underlying skin (similar to bandage designs.

This eliminates the need to roughly use scissors to cut out a circular shape and eliminates the need to carry scissors. Furthermore, having several of these rings 1201 functionally segmented from one another (between the perforations), if one ring segment ever fails, it would not result in an instant leak as would generally occur with most existing designs because each of the rings 1201 acts as its own adhesive barrier between the skin and material. Additional optional holes 1202 may be placed in and around these rings. These holes 1202 would allow additional breathability of the skin, but the holes would not be large enough to allow bodily fluids to leak out.

An additional embodiment may be an implant formed of a material which is stretchy/elastic so that it is configured to form itself around the stoma or to be molded by the user to fit abnormal (non-circular) stoma shapes. This solves the issue of existing designs which are tailored to fit only circular stoma shapes which is not always be the case. With the elastic design, a user may purchase a certain size of skin adhesive material which is slightly smaller than their stoma. This material may then be stretched around the full stoma, allowed to relax (close around the stoma), and then adhered to the skin. Similarly, a moldable material could be used to accommodate these abnormal anatomies. In this case, the user would be able to apply a paste or layer of material to the skin which would stick to the skin and the moldable material. Then, the user would be able to use this moldable material (akin to clay) to shape-form around their stoma to create a strong seal.

Current two-part bags consist of a snap-lock between the ostomy bag and the patch connected to the skin. In combination with the implants described above, if there were an embedded or built-in component to a stoma device/implant/attachment such as those described in the preceding Figures, alternate mechanical locking mechanisms including but not limited to twist-lock, vacuum seal, and lever-lock, may be utilized to connect the ostomy bag to the implant.

A final embodiment involves the use of electromagnets to maintain a reliable, easy-to-use connection between the ostomy management device and the skin/stoma. In this embodiment, a ring of ferromagnetic and biocompatible metal may be placed under the skin (or embedded within an implant such as those described in the preceding Figures) around the circumference of the stoma either as a solid metal ring or as a ring of beads to accommodate changes to the anatomy over time.

FIG. 13 shows a diagram 1300 of ferrous metal beads 1302 in a ring 1301 around a stoma. It should be understood that the ring 1301 may be embedded in the patient anatomy or within an implant. The stoma bag or waste receptacle designed with this embodiment may use either an electromagnet which may be engaged on or off, or a permanent magnet, to be able to form a connection between the two sections (inside the body and outside). This may be combined with other attachment methods such as an adhesive on a soft material to reduce pinch risks and improve overall comfort to the user.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. An ostomy device, comprising:
an implant shaped in a ring and sized for placement in or adjacent to a hole in an abdominal wall, the implant including a surface for contacting a lumen,
wherein the implant includes a first part including the surface for contacting the lumen, the first part, when placed in or adjacent to the hole in the abdominal wall, extending in a direction orthogonal to the abdominal wall to support the lumen, and
wherein the implant includes a region for attaching an ostomy bag.

17. The ostomy device of claim 16, wherein a cross section of the implant comprises a corner at a proximal end of the first part and a second part extending radially outward from the corner.

18. The ostomy device of claim 17, wherein, to form a stoma, the second part is inserted through the hole in the abdominal wall so that a top surface of the second part is brought into contact with an interior surface of the abdominal wall, the first part extends through the hole and protrudes a distance past the abdominal wall, and the lumen is brought through a center channel of the implant, wherein an inner surface of the first part contacts an outer surface of the lumen.

19. The ostomy device of claim 18, wherein the lumen is attached to the first part and the second part is attached to the abdominal wall.

20. The ostomy device of claim 17, wherein, to form a stoma, the second part is placed on an outer surface of the abdominal wall surrounding the hole in the abdominal wall so that a bottom surface of the second part is brought into contact with the outer surface of the abdominal wall, the first part extends a distance distally from the abdominal wall, and the lumen is brought through a center channel of the implant, wherein an inner surface of the first part contacts an outer surface of the lumen.

21. The ostomy device of claim 20, wherein the lumen is attached to the first part and the second part is attached to the abdominal wall.

22. The ostomy device of claim 16, wherein a cross section of the implant comprises a corner at a distal end of the first part, a second part extending radially outward from the corner and a distal part extending from the corner.

23. The ostomy device of claim 22, wherein, to form a stoma, the implant is inserted through the hole in the abdominal wall so that a top surface of the second part is brought into contact with an interior surface of the abdominal wall, the first part extends proximally, the distal part contacts tissue surrounding the hole in the abdominal wall, and the lumen is brought through a center channel of the implant, wherein an inner surface of the first part contacts an outer surface of the lumen and the lumen remains internal to the abdominal wall.

24. The ostomy device of claim 23, wherein the lumen is attached to the first part and the second part is attached to the abdominal wall and wherein the distal part comprises the bag attachment region within or adjacent to the hole in the abdominal wall.

25. The ostomy device of claim 16, wherein the implant comprises a distal part on a distal end of the first part.

26. The ostomy device of claim 25, wherein, to form a stoma, the implant is inserted through the hole in the abdominal wall so that the distal part contacts tissue surrounding the hole in the abdominal wall and an inner surface of the lumen is brought around the first part to an inner surface of the abdominal wall.

27. The ostomy device of claim 26, wherein the lumen is attached to the first part or to the inner surface of the abdominal wall, and wherein the distal part comprises the bag attachment region within or adjacent to the hole in the abdominal wall.

28. An adhesive material, comprising:
a first adhesive surface for placement on an area surrounding an entrance opening to an ostomy bag; and
a second adhesive surface for placement on a stoma or an implant integrated with the stoma,
wherein the adhesive material has multiple concentric circles of perforations to facilitate tearing by a user to size the adhesive material for an anatomy of the user comprising the stoma.

29. The adhesive material of claim 28, wherein the perforations provide breathing holes.

30. The adhesive material of claim 28, further comprising:
multiple breathing holes disposed between the circles of perforations.

31. A method for implanting an ostomy device, comprising:
placing an implant of the ostomy device in or adjacent to a hole in an abdominal wall, the implant shaped in a ring and including a surface for contacting a lumen, the implant including a first part including the surface for contacting the lumen, the first part, when placed in or adjacent to the hole in the abdominal wall, extending in a direction orthogonal to the abdominal wall to support the lumen, the implant comprising a region for attaching an ostomy bag; and
attaching the lumen to the implant to form a stoma.

32. The method of claim 31, wherein a cross section of the implant comprises a corner at a proximal end of the first part and a second part extending radially outward from the corner,
wherein, to form the stoma, the second part is inserted through the hole in the abdominal wall so that a top surface of the second part is brought into contact with an interior surface of the abdominal wall, the first part extends through the hole and protrudes a distance past the abdominal wall, and the lumen is brought through a center channel of the implant, wherein an inner surface of the first part contacts an outer surface of the lumen, and
wherein the lumen is attached to the first part and the second part is attached to the abdominal wall.

33. The method of claim 31, wherein a cross section of the implant comprises a corner at a proximal end of the first part and a second part extending radially outward from the corner,
wherein, to form the stoma, the second part is placed on an outer surface of the abdominal wall surrounding the hole in the abdominal wall so that a bottom surface of the second part is brought into contact with the outer surface of the abdominal wall, the first part extends a distance distally from the abdominal wall, and the lumen is brought through a center channel of the implant, wherein an inner surface of the first part contacts an outer surface of the lumen, and
wherein the lumen is attached to the first part and the second part is attached to the abdominal wall.

34. The method of claim 31, wherein a cross section of the implant comprises a corner at a distal end of the first part, a second part extending radially outward from the corner and a distal part extending from the corner,
wherein, to form the stoma, the implant is inserted through the hole in the abdominal wall so that a top surface of the second part is brought into contact with an interior surface of the abdominal wall, the first part extends proximally, the distal part contacts tissue surrounding the hole in the abdominal wall, and the lumen is brought through a center channel of the implant, wherein an inner surface of the first part contacts an outer surface of the lumen and the lumen remains internal to the abdominal wall,
wherein the lumen is attached to the first part and the second part is attached to the abdominal wall, and
wherein the distal part comprises the bag attachment region within or adjacent to the hole in the abdominal wall.

35. The method of claim 31, wherein the implant comprises a distal part on a distal end of the first part,
wherein, to form the stoma, the implant is inserted through the hole in the abdominal wall so that the distal part contacts tissue surrounding the hole in the abdominal wall and an inner surface of the lumen is brought around the first part to an inner surface of the abdominal wall, and
wherein the lumen is attached to the first part or to the inner surface of the abdominal wall, wherein the distal part comprises the bag attachment region within or adjacent to the hole in the abdominal wall.
